(19) United States
(12) Patent Application Publication
(10) Pub. No.: US 2010/0305330 A1
(43) Pub. Date: Dec. 2, 2010

(54) Process for preparing 2-alkyl-3-halo-6-nitrilpyridine and its carboxylic acid and ester derivatives

(75) Inventors: Heng E. Michael Su, (US); Yizu Wu, (US); Chunming Wu, (US); Haiyan Li, (US); Lee Alan DeWitt, Howell, MI (US)

Correspondence Address: Lee DeWitt, 1903 Oak Squire Lane, Howell, MI, 48855, US

(73) Assignee: Synergetica Changzhou Chemical Company, Changzhou (CN)

(21) Appl. No.: 12/455,425

(22) Filed: Jun. 2, 2009

(51) Int. Cl. C07D 213/803

(52) U.S. Cl. 546/327

(57) ABSTRACT

A process for preparing 2-alkyl-3-halo-6-nitrilpyridine and its carboxylic acid and ester derivatives by reacting 2-alkyl-3-halo-pyridine N-oxide with dimethyl sulfate, followed by reacting the adduct with alkaline cyanide to obtain the target molecule is disclosed. Further treatment of the nitrile with base to obtain the corresponding acid, and esterification are described as well. The process was scaled up to multi-kilogram level that provided satisfactory output.

### CROSS-REFERENCES TO RELATED APPLICATIONS

Organic Synthesis, Collective Volume 5, pp 269 (1962)

Organic Synthesis, Annual Volume 80, page 133 (2003)

Polish Journal of Chemistry, Volume 65(2-3), pp 289-295 (1991)

US Patent Application Publication, Pub. No.: US2002/0016470 A1, Feb. 7, 2002.

### STATEMENT REGARDING FEDERALLY FUNDED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

1. Technical Field

The invention relates to a novel process for preparing 2-alkyl-3-halo-6-nitrilpyridine and its carboxylic acid and ester by reacting 2-alkyl-3-halo-pyridine N-oxide with dimethyl sulfate, followed by reacting with alkaline cyanide to obtain the target molecule. 2-Alkyl-3-halo-6-nitrilpyridine and its carboxylic acid and ester are 2-Alkyl-3-halo-6-nitrilpyridine and its carboxylic acid and ester are pharmaceutically useful compounds in drug discovery projects. The above-mentioned molecules and derivatives can be used as intermediates in the synthesis of biologically active molecules that treat cardiovascular disorders.

2. Background Art

Usually the pyridine carboxylic acids are synthesized via pyridine alkyl group oxidation with strong mineral acids. The strong acid oxidation may very likely be non-selective to the alkyl groups on the pyridine ring, leading to low yield of the targeted carboxylic acids. For example, oxidation of a 3-methyl group on the pyridine ring could lead to nicotinic acid derivatives. To indirectly synthesize the pyridine carboxylic acid via piperidone is lengthy and economically disadvantageous. Therefore the identification of an efficient and scalable chemical process is necessary. Recently 3-bromo-2-methylpyridine was synthesized through the bromination of pyridine according to the literature. After removal of the undesired isomer, 5-bromo-2-methylpyridine, the enriched 3-bromo-2-methylpyridine was used in this invention as the starting material. Based on chemistry similar to that described in Organic Synthesis, Collective Volume 5, pp 269 (1962), the target molecules of this invention are synthesized as new compounds, which are not reported in the literature to date.

US Patent Application Publication, Pub. No.: US2002/0016470 A1, Feb. 7, 2002 described a new method to introduce a carbonyl group into the 2-position of pyridine ring. The method involves the formation of 2-lithio-5-halopyridine by reacting BuLi with 2,5-dihalopyridine in a non-coordinated solvent. The high selectivity of 2-lithiopyridine toward transformation to the formyl group and further to the corresponding carboxylic acid via mild oxidation is attractive, but the reaction requires low temperature at −78 and low concentration of precursor (<0.085M), making the large-scale preparation non-practical.

Therefore, this invention provides an industrially desirable process to prepare the 2-pyridine carboxylic acid and its derivatives.

### BRIEF SUMMARY OF THE INVENTION

In accordance with the present invention, a process of preparing 2-alkyl-3-halo-6-nitrilpyridine and its carboxylic acid and ester comprise the steps of
(a) preparation of N-Oxide of 2-alkyl-3-halo-pyridine with peracetic acid,
(b) adding dimethyl sulfate to form 1-methoxy-2-alkyl-3-halo-pyridinium methyl sulfate,
(c) reacting the adduct 1-methoxy-2-alkyl-3-halo-pyridinium methyl sulfate with alkaline cyanide to form the pyridine nitrile,
(d) hydrolysis of the nitrile with base to form the corresponding carboxylic acid
(e) esterification of the carboxylic acid yields the corresponding ester.

### BRIEF DESCRIPTION OF THE SEVERAL VIEWS OF THE DRAWING(S)

Not Applicable

### DETAILED DESCRIPTION OF THE INVENTION

The process of this invention can be used to prepare 3-halo-2-alkyl-6-cyanopyridine and 3-halo-2-alkyl-pyridine-6-carboxylic acid and its ester.

The starting material for this invention is 2-alkylpyridine. The bromination of 2-alkylpyridine with aluminum chloride as a Friedle-Crafts catalyst affords 3-halo-2-alkylpyridine and 5-halo-2-alkylpyridine. The latter is the major isomer in by-product distribution.

After removal of 5-halo-2-alkylpyridine, the enriched 3-halo-2-alkylpyridine is subject to oxidation with peracetic acid or hydrogen peroxide to form the corresponding pyridine N-oxide. With recrystallizations, 3-halo-2-alkylpyridine N-oxide can be purified up to 98% assay, which is pure enough for the successive steps.

The pyridine N-oxide was treated with dimethyl sulfate to obtain 1-methoxy-2-alkyl-3-halo-pyridinium methyl sulfate. 1-Methoxy-2-alkyl-3-halo-pyridinium methyl sulfate was treated with alkaline cyanide yielding 2-alkyl-3-halo-6-cyanopyridine that can be easily converted to the corresponding acid and its ester.

2-Alkyl-3-halo-6-cyanopyridine was combined with a mixture of water and alcoholic solution and brought to reflux. Monitoring by thin layer chromatography indicated the end of the reaction, and formation of 2-alkyl-3-halopyridine-6-carboxylic acid alkaline salts. Upon acidification with a mineral acid, 2-alkyl-3-halo-pyridine-6-carboxylic acid was crystallized from the solution. A filtration afforded pure acid. When 2-alkyl-3-halo-pyridine-6-carboxylic acid was treated in alcohol in the presence of a trace amount of mineral acid, the corresponding esters are formed in high yield.

The process of this invention is robust and amenable to scale up.

### EXAMPLES

### Example 1

### 3-Bromo-2-methylpyridine N-oxide synthesis

In a 5-l. four-necked round bottom flask equipped with agitation, a thermometer, a condenser and a dropping funnel, was placed 3-bromo-2-methylpyridine (110 grams, 1.18 moles) and dichloromethane (2 .L). The mixture was stirred, and per-acetic acid (900 grams, 20%, 2.37 moles) was added at such a rate that the temperature was maintained at 30° C. After the addition, the mixture was stirred for 3 hrs. at 20-30° C. The reaction was quenched with sodium sulfite solution when the raw material was detected at less than 2%.

To another 10-l. four-necked round bottom flask was added sodium sulfite (180 grams) dissolved in water (1100 grams). The mixture was stirred for 20 min., and the reaction mixture was added to the 10-l. flask at a rate such that the temperature did not exceed 30° C. After the addition, the mixture was stirred 1 hr. at 30° C. Then sodium hydroxide solution (320 grams, 50%) was added drop-wise into the mixture while the temperature was maintained at below 30° C. After phase separation, the aqueous layer was extracted with dichloromethane (130 grams). The extraction was repeated twice and the organic phases were combined, and its pH was adjusted to 14 with a sodium hydroxide solution (50%). Water (200 ml) was added as well. The organic phase was washed with brine, and concentrated by removal of the solvent. The residue was crystallized in ethyl acetate (400 grams). Off-white needle solid was obtained (155 grams, 69.5%).

### Example 2

### 3-Bromo-1-methoxy-2-methylpyridinium methyl sulfate synthesis

In a 1-l. three-necked round bottom flask equipped with a stirrer, a thermometer, and a 250-ml. pressure-equalizing dropping funnel fitted with a calcium chloride drying tube, was placed 3-bromo-2-picoline-1-oxide (188 grams, 1.0 mole). The stirrer was started at a slow rate, and dimethyl sulfate (126 grams, 1.0 mole) was added drop-wise at a rate such that the temperature of the reaction mixture slowly raised to between 80° C. and 90° C. and remained in this range throughout the addition. After the addition was completed, the mixture was heated for an additional 0.5 hrs. at 100° C. The obtained brown mixture (100%) was sealed in the flask and used directly in the next step.

### Example 3

### 5-Bromo-2-cyano-6-methylpyridine synthesis

In a 2-l. four-necked round bottom flask equipped with agitation, a dropping funnel, and a gas inlet adapter, was placed a solution of sodium cyanide (147 grams, 3.0 moles) dissolved in water (400 ml). While agitating, the apparatus was flushed with nitrogen for 1 hr. The solution in the flask was then cooled below 0° C. with an ice bath, and a solution of 3-bromo-1-methoxy-2-methylpyridinium methyl sulfate (314 grams, 1.0 mole) dissolved in water (300 ml) was added drop-wise at a rate that the temperature was maintained between 0-5° C. The dropping funnel and the thermometer-adapter were then quickly removed and replaced by stoppers, and the flask was allowed to stand in a refrigerator overnight (16 hrs.). The flask, containing the crude nitrile, was removed from the refrigerator and the contents were stirred at room temperature for 4 hours. After filtration, the obtained brown crude was washed with water to pH 7 then recrystallized with ethyl acetate and activated carbon. Off-white solid (124 grams, 63%) was obtained. The purity of the product was >98%.

### Example 4

### 3-Bromo-2-methylpyridine N-oxide synthesis

To a 2000-l. glass-lined reactor was added 3-bromo-2-methylpyridine (80 Kg, 316.3 moles) and dichloromethane (778 Kg). The mixture was stirred, and per-acetic acid (250 Kg, 658 moles, 20%) was added to the vessel at such a rate that the temperature was maintained below 30° C. After the addition, the mixture was stirred for an additional 6 hrs. at 20-30° C. The reaction was quenched when the raw material was detected at less than 1%, by adding to the reactor sodium sulfite solution (820 Kg, 15%) at a rate that the temperature did not exceed 30° C. After the addition, the mixture was stirred for 1 hr. at 30° C. and tested for the presence of peroxide. If peroxides are detected, additional sodium sulfite was added until no more peroxide existed. Sodium hydroxide solution (220 Kg, 50%) was added drop-wise into the mixture while the temperature was maintained below 30° C. After phase separation, the aqueous layer was adjusted with sodium hydroxide solution (80 Kg, 50%) to pH 14. Dichloromethane (372 Kg) was added as well. The extraction was repeated once and the organic phases were combined. The organic phase was washed with sodium chloride solution (74 Kg) dissolved in water (500 Kg), and concentrated by removal of the solvent. The residue was crystallized with ethyl acetate (120 Kg). The obtained rude material (60 Kg) was recrystallized with dichloromethane:ethyl acetate=1:3 (200 Kg). The product (40.5 Kg, 215 moles, 68%) was obtained as white needle crystals. The purity was >98%.

### Example 5

### 3-Bromo-1-methoxy-2-methylpyridinium methyl sulfate synthesis

In a 5-l. four-necked flask equipped with agitation and a thermometer was placed 3-bromo-2-picoline-1-oxide (2 Kg, 10.64 moles). The stirrer was started at a slow rate, and dimethyl sulfate (1.34 Kg, 10.64 moles) was added drop-wise at a rate such that the temperature of the reaction mixture slowly rised to between 80° C. and 90° C. and remained in this range throughout the addition. After the addition was completed, the mixture was heated for an additional 0.5 hrs. at 100° C. The obtained brown mixture (100%) was sealed in the flask and used directly in the next step.

### Example 6

### 5-Bromo-2-cyano-6-methylpyridine synthesis

Into a 500-l. glass-lined reactor was placed a solution of sodium cyanide (27.6 Kg, 563 moles) and water (75 Kg). To another 200-l. glass-lined reactor was added 3-bromo-1-methoxy-2-methylpyridinium methyl sulfate (59.67 Kg, 190 moles) and water (57 Kg). The obtained solution was added to the 500-l. reactor at a rate that the temperature was maintained at 0-5° C. After the addition, the mixture was stirred for 1 hr. and then was allowed to stand for 16 hrs. at a temperature below 0° C. Then the temperature of the contents was slowly raised to room temperature and stirred for another 4 hrs. After centrifugation, the obtained brown crude was washed with water to pH 7. The purity of the wet cake (60.5 Kg) was 81%.

To a 300-l. glass-lined reactor was added the wet cake, ethyl acetate (352 Kg), and activated carbon (9.5 Kg). The mixture was agitated and heated to reflux. Then it was slowly cooled down to room temperature and filtered. The filtration was distilled under vacuum until 45 Kg of the residue remained. The residue was crystallized with hexane (10 Kg). After centrifugation and drying, off-white solid (25.5 Kg, 129.4 moles) was obtained, and its purity was >98%. The yield was 68%.

### Example 7

### 5-Bromo-6-methyl-2-pyridine carboxylic acid synthesis

In a 250-ml. four-necked round bottom flask equipped with agitation, a thermometer, and a condenser, was placed 3-bromo-2-methyl-6-cyanopyridine (25 grams, 0.127 moles) followed by sodium hydroxide (14.3 grams, 0.358 moles) and methanol (143 ml, 75%). The mixture was stirred and heated to reflux (66° C.) for 1.5˜2 hrs. The reaction was stopped when the raw material was detected at less than 1%. Solvent was removed under vacuum pressure at a temperature below 50° C. After the concentration, water (100 ml) was added and the mixture was cooled to 0° C. Concentrated hydrochloric acid was added to adjust the pH to 7, resulting in precipitation of the product. After filtration and drying, white solid (24.5 grams, 0.113 moles) was obtained. The purity of the product was >98%, and the yield was 89%.

### Example 8

### Methyl 5-bromo-6-methyl-2-pyridine carbonate synthesis

In a 250-ml. four-necked round bottom flask equipped with agitation, a thermometer, a condenser, and a dropping funnel was placed 5-bromo-6-methyl-2-pyridine carboxylic acid (10 grams, 0.046 moles) and methanol (44.1 grams, 1.378 moles). The mixture was agitated and thionyl chloride (8.5 grams, 0.071 moles) was added drop-wise while the temperature was maintained between 20˜30° C. After the addition, the mixture was heated to 55˜65° C. The reaction was stopped when the raw material was detected at less than 2%. The solvent was removed by evaporation. MTBE (93 grams) was added to dissolve the crude and the MTBE solution was poured into water at 0˜5° C. After phase separation, the aqueous layer was extracted with MTBE (50 ml). The organic phases were combined, washed with brine and concentrated. White powder (6.9 grams, 0.03 moles) was obtained. The purity of the product was >98%, and the yield was 65%. ¹H NMR (DMSO-d₆) δ 2.6 (s, 3H), 3.8 (s, 3H), 7.75 (d, J=0.05 Hz, 1H), 8.22 (d, J=0.05 Hz, 1H).

### Example 9

### 5-Bromo-6-methyl-2-pyridine carboxylic acid synthesis

Into a 300-l. stainless steel reactor was placed 3-bromo-2-methyl-6-cyanopyridine (18 kg, 91.37 moles), sodium hydroxide (10 kg, 250 moles), water (26 kg) and methanol (59 kg). The mixture was stirred and heated to reflux (66° C.) for 1.5˜2 hrs. The reaction was stopped when the raw material was detected at less than 1%. Solvent was removed under vacuum at a temperature below 50° C. After concentration, water (72 kg) was added and the mixture was cooled to 0° C. Conc. sulfuric acid (105 kg) was added to adjust the pH to 7, precipitating the product. After centrifugation and drying, white solid (17.8 kg, 82.3 moles) was obtained. The purity of the product was >98%, and the yield was 90%.

### Example 10

### Methyl 5-bromo-6-methyl-2-pyridine carbonate synthesis

Into a 500-l. glass-lined reactor was placed 5-bromo-6-methyl-2-pyridine carboxylic acid (30 kg, 138.9 moles) and methanol (133 kg, 4156 moles). The mixture was agitated and thionyl chloride (36.8 kg, 309.2 moles) was added while the temperature was maintained between 20˜30° C. After the addition, the mixture was heated to 55˜65° C. for 2 hrs. The reaction was stopped when the raw material was detected at less than 2%. Then solvent was removed by concentration at a temperature below 40° C. MTBE (280 kg) was added and the mixture was stirred for another 30 min. to dissolve the crude. Then the MTBE solution was transferred to one drum. To the above reactor was added water (300 kg). Then the solution containing the crude was pumped to the reactor while maintaining the reactor contents at 0˜5° C. and stirred for 1 hr. After phase separation, the aqueous layer was extracted with MTBE (60 kg). The organic phases were combined, and concentrated at a temperature below 40° C. Hexane (60 kg) was added to the residue. The obtained solid was stirred, centrifuged and dried. White powder (22.36 kg, 97.2 moles) was obtained. The purity of the product was >98%, and the mole yield was 70%.

### SEQUENCE LISTING

Not Applicable

What is claimed is:

1. A process to prepare 2-alkyl-3-halo-6-cyanopyridine and 2-alkyl 3-halopyridine-6-carboxylic acid and esters of 2-alkyl-3-halopyridine-6-carboxylic acid comprised of the following steps:
(a) preparation of N-Oxide of 2-alkyl-3-halo-pyridine with peracetic acid,
(b) adding dimethyl sulfate to form 1-methoxy-2-alkyl-3-halo-pyridinium methyl sulfate,
(c) reacting the adduct 1-methoxy-2-alkyl-3-halo-pyridinium methyl sulfate with alkaline cyanide to form the 2-alkyl-3-halo-6-nitrilpyridine,
(d) hydrolysis of the 2-alkyl-3-halo-6-nitrilpyridine with base to form the corresponding 2-alkyl-3-halopyridine 6-carboxylic acid
(e) esterification of the 2-alkyl-3-halopyridine 6-carboxylic acid to yield the corresponding 2-alkyl-3-halopyridine 6-carboxylic acid ester.

2. A process according to claim 1 wherein the alkyl group is methyl, ethyl, propyl, isopropyl, butyl, isobutyl, t-butyl, pentyl, hexyl, heptyl, octyl, nonyl, decyl, dodecyl, cyclopropyl, cyclopentyl, cyclohexyl or benzyl

3. A process according to claim 1 wherein the ester group is methyl, ethyl, propyl, isopropyl, butyl, isobutyl, t-butyl, pentyl, hexyl, heptyl, octyl, nonyl, decyl, dodecyl, cyclopropyl, cyclopentyl, cyclohexyl or benzyl

4. A process according to claim 1 wherein the halo group is fluoro, chloro, bromo or iodo
